(19) United States
(12) Patent Application Publication
(10) Pub. No.: US 2019/0011426 A1
(43) Pub. Date: Jan. 10, 2019

(54) Nitrite detection device for detecting nitrite concentration

(71) Applicant: Soochow University, Taipei City (TW)

(72) Inventors: Mei-Lin Ho, Taipei City (TW); Kuen-Yuh Chung, New Taipei City (TW); Jian-Fan Zhu, Yuanlin City (TW); Yi-Chieh Chou, Taipei City (TW); Chu-Ting Chan, New Taipei City (TW); Sheng-Wei Pan, Taipei City (TW)

(21) Appl. No.: 15/804,325

(22) Filed: Nov. 6, 2017

(30) Foreign Application Priority Data
Jul. 7, 2017 (TW) 106122802

(51) Int. Cl. G01N 33/493; G01N 33/52; G01N 33/487; G01N 27/327; G01N 27/40

(52) U.S. Cl. CPC G01N 33/493; G01N 33/523; G01N 27/40; G01N 27/327; G01N 33/48707

(57) ABSTRACT

Present invention relates to a nitrite detection device comprising of an electrochemical test strip and a colorimetric test paper, said electrochemical test strip is consisting of an insulating substrate and an electrochemical detection structure placed in the insulating substrate. When a fluid sample enters the sensing region and detecting region of the electrochemical detection structure, it will undergo an electrochemical reaction. Detecting the change of the electric signals to accurately calculate the nitrite concentration in the fluid sample, while examining whether a positive nitrite response is present by using the colorimetric test paper. Present invention discloses a combined nitrite detection device that has the advantages of real-time semi-quantification and accurate quantification of nitrite concentration via electrochemical reaction.

### BACKGROUND OF THE INVENTION

### 1. Field of the Invention

Present invention relates to a nitrite detection device, in particular, a combined nitrite detection device which can simultaneously perform semi-quantitation and accurate quantitation of nitrite concentration in a sample by using both the colorimetric method and electrochemical method.

### 2. Description of the Prior Art

The use of nitrite in meat has lasted a long time and the purpose of addition of this additive is to fix meat color, provide special flavor, inhibit rancidity of fatty acids and inhibit microbial growth. Nonetheless, nitrite has become the source of certain serious problems and the most notable one is nitrite may react with secondary amines in the meat and produce carcinogenic nitrosamines. In animal studies, nitrosamines have strong hepatoxicity and will cause hepatitis, liver cirrhosis as well as oral cancer, esophageal cancer, nasal cancer, tracheal cancer, lung cancer, liver cancer and pancreatic cancer.

In addition, fruits and vegetables may contain excessive amount of nitrites if planted in soil containing excess nitrites, and excessive intake of nitrites through ingestion of drinking water or drinks contaminated with nitrites may lead to denaturation of hemoglobin which will reduce the oxygen-carrying capacity of hemoglobin and cause hypoxia, weakness and rapid heartbeat, or central nervous system decline, headache and difficulty breathing in serious cases, or even shock, coma and death. Ingestion of excessive amount of nitrite in pregnant women may lead to higher risk of pregnancy complications such as anemia, abortion, premature birth or preeclampsia.

Also, in recent years, bacterial infection and urinary infection are commonly found in people of all ages, among which urinary infection is mostly found in young children, pregnant women and elderly people. Upper urinary tract infection will cause kidney scarring, hypertension and terminal renal dysfunction. At present, urinary tract infection is usually detected by urine test and bacterial infection can be detected by blood, that is, analysis and determination of the presence of nitrites in the blood or urine for detection of specific bacterial infection.

The routine detection methods used at present include colorimetric test paper, chromatographic quantification and optical quantification.

Taiwan Patent Number M516160 provides a nitrite detection device, consisting of a sampling module, a reactive module, a detection module, a first transmission module, an analysis module, a second transmission module and a display module, wherein the reactive module reacts with the sample and gives a result that corresponds to the amount of nitrites in the sample, the analysis module performs calculation to give the value of generated nitrites, however, this device is not for single-use and thus has the risk of contamination and it is not a portable device.

The U.S. Pat. No. 5,776,715 provides an optical detector for detecting nitrates or nitrite ions and is consisting of a reductase, a co-factor, a photoluminescent component and a transducer, and said transducer is comprising of a photoelectrical detector, said reductase will reduce nitrate to nitrite ions while the cofactor being oxidized by nitrite ion, oxidation of this cofactor will allow the photoluminescent component to produce photons and the amount of output photons will be analyzed by the photodetector to quantify the concentration of nitrate or nitrites. However, this device generates lights via oxidized cofactor, which is an indirect method of measurement and is not a direct method for quantification.

China Patent Number 103235093 provides a fast chromatographic detection method for detecting nitrites in the blood and urine and is comprising of the following steps: preparation of nitrite standard colorimetric solutions, detection of nitrites in the blood or urine, mix of the sample, sodium hydroxide solution and zinc sulfate thoroughly, addition of acetonitrile before centrifugation, collection of the supernatant before addition of Griess reagent, addition of non-ion surfactant before centrifugation and chromatographic separation, and finally comparison of the color of the upper layer surfactant of the solution with the prepared standard colorimetric solutions to determine its concentration. This chromatographic method requires multiple pre-treatment of the sample before analysis and cannot analyze un-treated samples.

In terms of the common methods for detecting nitrites, chromatographic quantification requires longer processing time and is not popular, in addition, optical quantification and chromatographic quantification need expensive equipment and more samples and these samples need to be pre-treated to prevent follow-up interference with absorbance. The chromatographic method requires numerous pre-treatment processes and complicated steps for solution reaction and this is a semi-quantitative method and is more difficult to accurately interpret the test result. As for colorimetric test paper, the color will be affected by many factors such as sample concentration, reaction time, red blood cell or bacterial contamination and cannot accurately determine nitrite concentration in the body. In addition, sample color may easily interfere with the resulting color. Moreover, for the detection device for detecting nitrite concentration (for example, Taiwan Utility Patent Number M516160), the concentration of nitrites in the sample is determined by an electrochemical method but addition of other reaction reagents is required during the process of reaction to allow generation of electric current and electrical resistance via nitrite reaction in the sample for analysis and therefore this type of device cannot provide a real-time result. Due to popularization of at-home detection device, developing a detection device that has a simplified construction, is easy to operate and portable, and provides both semi-quantification and quantification functions is one of the important tasks for the field of this invention.

### SUMMARY OF THE INVENTION

Based on the tasks mentioned above, according to the nitrite detection device of present invention, said device is a combined test strip equipped with a test paper capable of semi-quantitative analysis with short reaction time and an electrochemical test strip that provides accurate quantification, does not need extra reaction reagents and offers improved accuracy of the test result, is portable and can be used without professional training.

In one aspect, present invention provides a test strip that provides the functions of both semi-quantification and accurate quantification of nitrite concentration in the sample, the test paper can be used for semi-quantitative analysis of the sample while the concentration can be calculated via change of the electric signals such as electric current or electrical resistance by using the electrochemical test strip.

In another aspect, present invention provides a nitrite detection device that offers stable and reproducible test results by taking the advantages of the reaction reagents that specifically react with nitrites to generate an electrochemical reaction and said reaction has long-term stability in the air and the test result will not be misinterpreted due to interference from the sample color.

In the other aspect, present invention provides a portable and real-time nitrite detection device, its size is small and contains the reagents that can react with nitrites in the sample and hence can be used directly without addition of extra reaction reagents.

To achieve the abovementioned goals and effects, present invention provides a nitrite detection device, comprising of an electrochemical region and a visual colorimetric region, said electrochemical region is installed with an electrochemical test strip and said visual colorimetric region is equipped with multiple colorimetric test papers.

Wherein the electrochemical test strip is comprising of: an insulating substrate, an electrochemical structure, a first reaction layer and a second reaction layer, said insulating substrate has a first surface and a second surface and said second surface is on the back of the first surface; the electrochemical detection structure is consisting of a sensing region which is the reaction area for fluid sample, a detecting region for electrochemical reaction of the product in the sensing region, a conductive region for transmission of the product generated in the sensing region to the detecting region, said first layer covers the sensing region and is coated with a first reactive material, said first reactive material is consisting of a buffer solution and a nitrite reaction substance; said second reaction layer covers the detecting region and is coated with a second reactive material, said second reactive material consists of a surfactant and an electron transport material; when a fluid sample enters the sensing region and reacts with the first reactive material of the first reaction layer and enters the detecting region through the conductive region to react with the second reactive material of the second layer and undergoes an electrochemical reaction, change of the signal of electric current or electrical resistance caused by the amount of the electrochemical product generated in the detecting region will be used to calculate the concentration of nitrites.

According to the invention, the insulating substrate is selected from the group consisting of polycarbonate, polyester, polyether, polyamide, polyurethane, polyimide, polypropylene, polyethylene, polyvinyl chloride, glass, glass fiber, cotton fiber, silicon and aluminum dioxide.

According to the invention, said buffer solution is selected from the group consisting of phosphate, citrate and acetate buffer solution.

According to the invention, said surfactant is selected from the group consisting of Tween-20, Tween-80, Triton X-100, Mega8 and Polyvinylpyrrolidone (PVP).

According to the invention, said nitrite-reactive substance is p-aminobenzenesulfonamide.

According to the invention, said electron transport materials are conductive metal and N-1-naphthylethylenediamine.

According to the invention, said conductive metal is selected from the group consisting of gold, silver and copper.

According to the invention, said electrochemical test strip may further comprise an electrode structure covering the first surface of the insulating substrate, said electrode structure is consisting of a working electrode and a reference electrode, wherein the working electrode is a conductive colloid and said conductive colloid is selected from the group consisting of copper colloid, nickel colloid, silver colloid, gold colloid, platinum colloid, silver-carbon colloid and carbon colloid.

According to the invention, said working electrode is gold, silver, copper, platinum or graphite and said reference electrode is a calomel electrode or a silver/silver chloride electrode.

According to the invention, said electrochemical test strip further comprises a hydrophilic filter membrane covering the sample guide groove of the second substrate and is used for filtration of the impurities in the fluid sample.

Wherein, said colorimetric test paper is comprising of: a sample test paper, a negative control region and a positive control region, said sample test paper is used to detect the same fluid sample of the electrochemical strip, said negative control region shows colorization of a sample that does not contain nitrites and the positive control test paper represents colorization of a sample containing known nitrites.

As stated above, the nitrite detection device of present invention is a combined detection device for detecting nitrites, in practical operation, adding adequate amount of the fluid sample to the sensing region of the electrochemical test strip to allow reaction of the first reactive material of the first layer with the fluid sample, which then enters the detecting region via the conductive region to react with the second reactive material of the second layer and undergoes an electrochemical reaction. Change of the signals of the electric current or electrical resistance caused by the amount of the electrochemical product generated in the detecting region will be used to calculate the concentration of nitrites. In addition, taking adequate amount of the fluid sample and adding the sample to the sample test paper, the negative control region and the positive control region are used as references for color change of the test paper region of this sample. Because the reaction time required for colorimetric test paper and the fluid sample is shorter than the time required for the reaction of the electrochemical strip, the result of the colorimetric test paper can be obtained quickly to determine whether the fluid sample gives a positive or negative result. If the sample color causes a false-positive result or the extreme low concentration of nitrites causes a false-negative result, the electrochemical test strip can be sued for quantitative analysis to accurately determine the nitrite concentration in the fluid sample and interpretation of the result is not affected by sample color. Moreover, said electrochemical test strip can be placed in sanitary pads and the lining of panties or diaper for real-time detection of nitrites in the urine of young children or elderly people for early detection of urinary tract infection.

In conclusion, the nitrite detection device of present invention has following advantages:
1. This detection device provides the functions of real-time semi-quantification and accurate quantification of the nitrite concentration in a sample by colorimetric method and electrochemical method, respectively.
2. The test result of the electrochemical test strip of this detection device is stable and can avoid misinterpretation caused by sample color.
3. The reaction layer of the electrochemical test strip and the colorimetric test paper of this detection device contain a reagent that can specifically react with nitrites and the device is ready for use without the need of additional reaction reagents and thus meets the requirements for an-home use.
4. Immediate use of the electrochemical test strip of this detection device after opening is not necessary and the strip can be placed in sanitary pads and the lining of panties or diaper or be tested with urine samples obtained from drainage bag or catheter to provide the convenience of real-time detection.
5. This detection device has higher sensitivity and lower limit of detection when compared to commercial test papers available in the market and only a few bacteria are required for detection of urinary tract infection.

### BRIEF DESCRIPTION OF THE DRAWINGS

FIG. 1 is the schematic diagram of the nitrite detection device of this invention.

FIG. 2 is the schematic diagram of the electrochemical test strip.

FIG. 3 is the schematic diagram of the electrode-containing electrochemical test strip.

### SYMBOLS AND KEY ELEMENTS

1 Nitrite detection device

1a Electrochemical measurement region

1b Visual colorimetric region

10 Electrochemical test strip

100 Insulating substrate

1000 First surface

101 Electrochemical detection structure

101a Detecting region

101b Conductive region

101c Sensing region

102 First reaction layer

103 Second reaction layer

104 Electrode structure

1040 Working electrode

1041 Reference electrode

11 Colorimetric test paper

110 Sample test paper

111 Positive control region

112 Negative control region

### DETAILED DESCRIPTION OF THE PREFERRED EMBODIMENT

To fully understand the purpose, features and functions of present invention, following examples are provided with embodiments to explain the details of present invention.

To ensure the consistency of the technical content of this invention, the definitions of terms are herein provided:

“Semi-quantitative analysis” herein refers to the analytical method for measuring the approximate content of an component in a material for testing, for example, intermediate control in the process, identification of whether the toxic exceeds the lethal dose, whether a certain ingredient exceeds the statutory standard to further select an appropriate concentration for follow-up accurate quantification.

“Quantitative analysis” herein refers to an analytical method for confirming the accurate content of a certain component of a material for testing based on its physical or chemical properties, e.g. light absorption, fluorescence, conductance by using specific equipment or devices, e.g. electrophoresis, chromatograph.

“Working electrode” herein refers to the electrode that is used to analyze the concentration of a specific substance in a fluid sample and shows significant change during the detection process of the electrochemical test strip and this electrode is not limited to an anode or a cathode. Depending on the reaction occurs at the electrode, an oxidation reaction at the electrode is called the anode, on the other hand, a reduction reaction at the electrode is called the cathode.

“Reference electrode” herein refers to an electrode with a potential that has stability and reproducibility and can be used as a reference for measuring the potential of other electrodes, that is, providing a standard potential and its potential will not vary with changes of the composition and concentration of the measurement system.

“Fluid sample” herein refers to the fluid material for testing that reacts with the reaction material of the reaction layer and undergoes chemical reaction, and its source can be urine, blood, drinking water, vegetable juice, milk or other fluids.

“Hydrophilic” herein refers to a chemical species or subgroup having a high affinity for water or aqueous solutions. Thus, a hydrophilic compound may be attached to, dissolved in or absorbed by water or an aqueous solution.

“Approximately” herein refers to a ±20%, or preferably ±10% of a specified value.

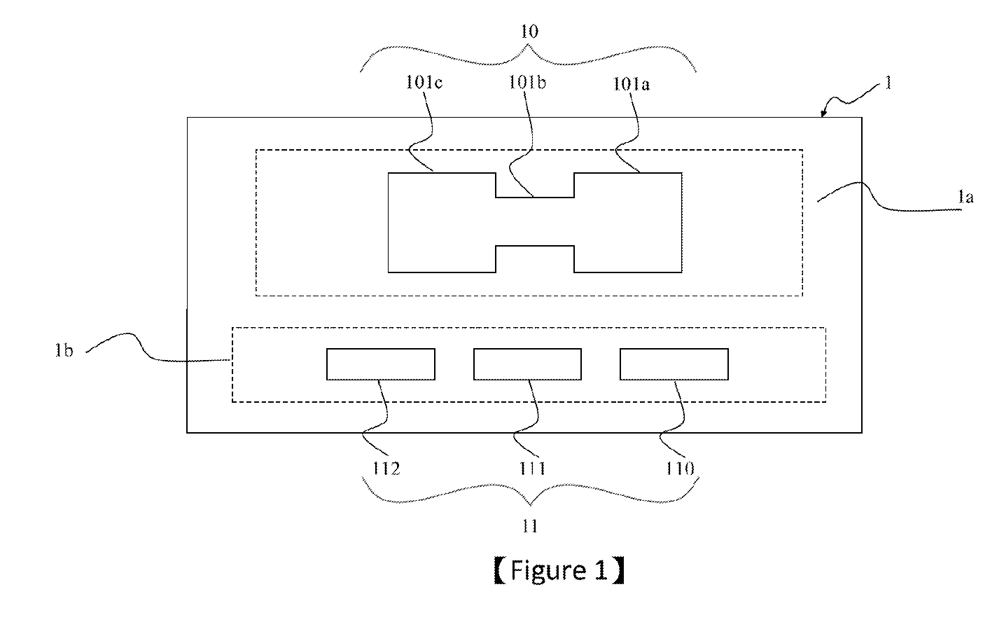

Please refer to FIG. 1, the first example of the nitrite detection device of present invention. In this example, said nitrite detection device 1 includes: an electrochemical measurement region 1a and a visual colorimetric region 1b, wherein said electrochemical measurement region 1a is equipped with a electrochemical test strip 10, said visual colorimetric region 1b is equipped with a colorimetric test paper 11, comprising of a sample test paper 110, a positive control region 111 and a negative control region 112, said negative control region 112 is used to present colorization of a sample that does not contain nitrites, said positive control region 111 is used to present colorization of a sample containing nitrites, said positive control region 111, said negative control test paper 112 and said sample test paper 110 are separated by a certain distance or waterproof protection to avoid the fluid sample of the sample test paper 110 enters the positive control region 111 and negative control region 112 and results in miss interpretation of the distance.

While conducting the fluid sample analysis, taking adequate volume and adding to the sample test paper 110 and electrochemical test strip 10 simultaneously or in sequential order, the reaction time of each colorimetric test paper 11 is approximately 5˜10 minutes, not longer than 30 minutes, so as to avoid miss interpretation due to prolonged reaction time. According to the concentration range of nitrites displayed by the sample test paper 110, the reaction time of the electrochemical test strip 10 is approximately 20 minutes. Next, determine the potential change of the electrochemical test strip 10 which can detect the signal of an electric current or electrical resistance to calculate nitrite concentration of the fluid sample. Because the reaction time of the colorimetric test paper 11 is shorter, it is preferable to obtain whether the fluid sample shows a positive nitrite response and its test result shows a certain concentration range before comparing the result with the quantified nitrite concentration measured by the electrochemical test strip 10 to further determine whether the measured concentration falls within the concentration range indicated by the test paper and avoid false positive caused by sample color or false negative caused by extremely low concentration of nitrites and to improve the credibility of the result.

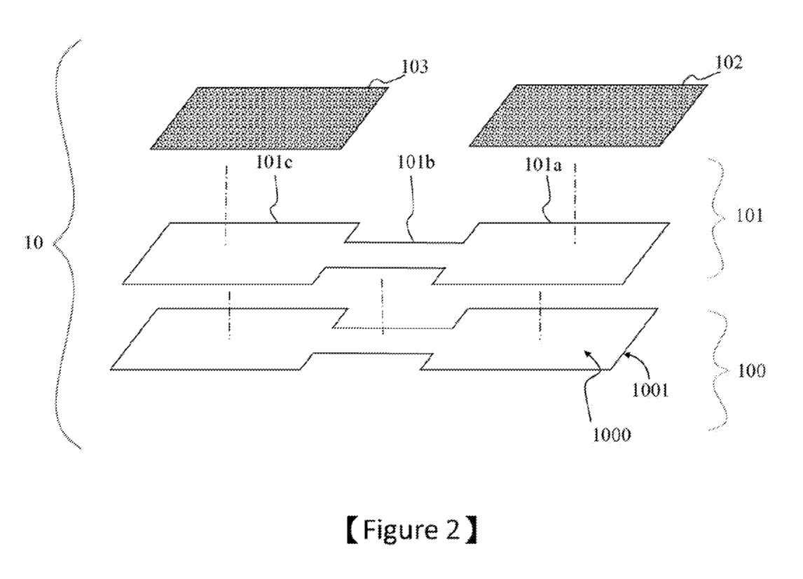

Please refer to FIG. 2, the second example of the nitrite detection device of present invention. FIG. 2 is a schematic diagram of the electrochemical test strip 10, and in this example, said electrochemical test strip 10 includes: an insulating substrate 100, an electrochemical detection structure 101, a first reaction layer 102 and a second reaction layer 103.

Wherein the insulating substrate 100 has a first surface 1000 and a second surface 1001 on the back of the first surface (FIG. 2), and said electrochemical test strip 101 is installed on the first surface 1000 of the insulating substrate 100, consisting of a detecting region 101a, a sensing region 101c, and a conductive region 101b installed between the detecting region 101a and the sensing region 101c, said first reaction layer 102 covers the sensing region 101c and is coated with a first reaction material, said first reaction materials include a buffer solution and a nitrite reaction material, said second reaction layer 103 covers the detecting region 101a and is coated with a second reaction material, said second reaction materials include a surfactant and an electron conductive substance, wherein a fluid sample enters the detecting region 101a and reacts with the first reaction material of the first reaction layer and undergoes a chemical reaction before entering the detecting region 101a via the conductive region 101b and reacts with the second reaction material of the second layer and undergoes an electrochemical reaction. The concentration of nitrites of the fluid sample is calculated according to the product generated from the electrochemical reaction.

Wherein one of the preferred example of the insulating substrate 100 is a soft material, for example, insulating high-molecular weight materials such as ceramic materials, glass or glass fibers. Examples of the insulating substrate 100 include, but are not limited to, polycarbonate, polyester, polyether, polyamide, polyurethane, polyimide, polypropylene, polyethylene, polyvinyl chloride, glass, glass fiber, cotton fiber, silicon dioxide, aluminum dioxide and the like.

Wherein the buffer solution contained in the reaction material includes, but is not limited to, phosphate buffer, citrate buffer, acetate buffer, etc. Examples of said surfactants include, but are not limited to, Tween-20, Tween-80, Triton X-100, Mega8, Polyvinylpyrrolidone (PVP). Examples of the nitrite-reactive substance are p-aminobenzenesulfonamide, and the examples of the electron-transporting material are conductive metals and N-1-naphthylethylenediamine. At a constant potential, the nitrite ion in the fluid sample undergoes an electrochemical reaction with the p-aminobenzenesulfonamide, N-1-naphthylethylenediamine and the electrically conductive metal to produce an electrical change.

Wherein the electrochemical structure 101 can be divided into three compartments, including a detecting region 101a, a sensing region 101c and a conductive region 101b installed between the detecting region 101a and the sensing region 101c. Said sensing region 101c refers to the region installed with the first reaction layer 102 in the electrochemical detection structure 101 for electrochemical reaction of the fluid sample and generate an electrical change. The product generated by the chemical reactions occurs in the sensing region 101c is delivered to the detecting region 101a via the conductive region 101b. In practical operation, the detecting region 101a will produce an electric change and detect the signal of electric current or electrical resistance for calculation and to give the nitrite concentration in the fluid sample.

Wherein one of the preferred example of the electrochemical detection structure 101, the conductive region 101b can be further installed with an insulating layer (not shown) and said insulating layer directly covers the electrode structure 101 located in the conductive region 101b.

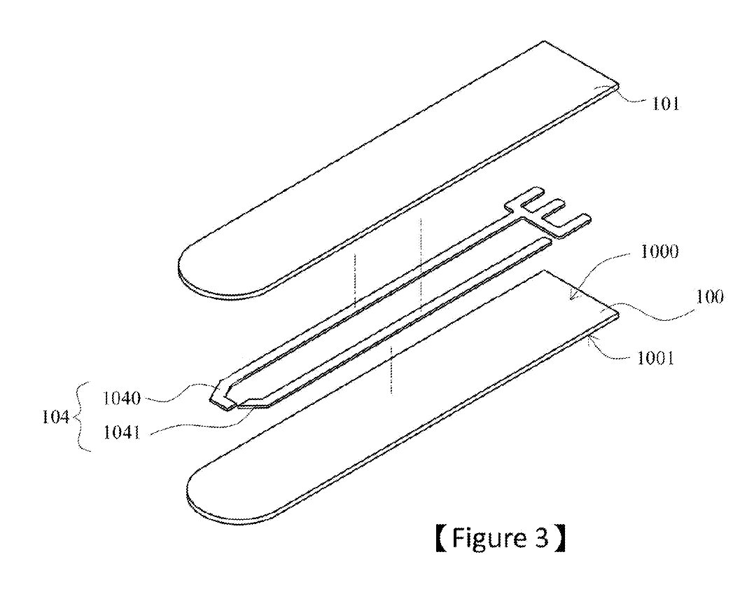

Please refer to FIG. 3, the third example of the nitrite detection device of present invention. In this example, the electrochemical test strip 10 is comprising of: an insulating substrate 100, an electrochemical detection structure 101, a first reaction layer 102, a second reaction layer 103, an electrode structure 104 and a hydrophilic filter membrane.

Wherein the insulating substrate 100 has a first surface 1000 and a second surface 1001 on the back of the first surface 1000, said electrochemical detection structure 101 can be divided into three compartments, including a detecting region 101a, a sensing region 101c and a conductive region 101b installed between the detecting region 101a and the sensing region 101c. Said sensing region 101c refers to the region installed with the first reaction layer 102 in the electrochemical detection structure 101 for electrochemical reaction of the fluid sample and generate an electrical change. Said electrode structure 104 is installed on the first surface 1000 of the insulating substrate 100 and is consisting of a working electrode 1040 and a reference electrode 1041; said hydrophilic filter membrane covers the sensing region 101c, when a fluid sample such as urine, blood, drinking water, milk, vegetable juice or other fluids touches the hydrophilic filter membrane, the impurities in the fluid sample will be filtered and removed and then react with the material of the first reaction layer 102 and undergoes the reaction.

Wherein one of the preferred example of the insulating substrate 100 is a soft material, for example, insulating high-molecular weight materials, or a hard material such as ceramic materials, glass or glass fibers. Examples of the insulating substrate 100 include, but are not limited to, polycarbonate, polyester, polyether, polyamide, polyurethane, polyimide, polypropylene, polyethylene, polyvinyl chloride, glass, glass fiber, cotton fiber, silicon dioxide, aluminum dioxide and the like.

Wherein the electrode structure 104 in a preferred example, the working electrode 1040 and the reference electrode 1041 are a conductive colloid, said conductive colloid is a resin colloid containing metal conductor or carbon particles and its materials include, but are not limited to, copper colloid, nickel colloid, silver colloid, gold colloid, platinum colloid, silver-carbon colloid, carbon colloid, and the colloid is printed onto the insulating substrate 100 via screen printing. In addition, to increase the conductivity of the conductive colloid, plasma cleaning can be used to further remove the impurities in the conductive colloid after finishing printing.

Wherein, the electrode structure 104 in another preferred example, the working electrode 1040 can be metal, silver, copper, platinum or graphite, wherein the electrical conductivity of gold is approximately 45.2×10⁶ S/m, the electrical conductivity of copper is approximately 59.6×10⁶ S/m and the electrical conductivity of silver is approximately 63.01×10⁶ S/m and hence the preferred one is silver and the reference electrode 1041 can be a calomel electrode or a silver/silver chloride electrode.

Wherein the buffer solutions contained in the reactive material include, but are not limited to, phosphate buffer, citrate buffer and acetate buffer solution and are primarily used to maintain the pH value within a certain range, the examples of said surfactant include, but are not limited to, Tween-20, Tween-80, Triton X-100, Mega8, Polyvinylpyrrolidone (PVP). In general, the concentration of surfactant is between 0.001% (w/v) and 10% (w/v), preferably between 0.1 and 5% (w/v), the example of said nitrite reactive material is p-aminobenzenesulfonamide, the examples of the electric conductor are conductive metals and N-1-naphthylethylenediamine, said conductive metal can be gold, silver, copper or other conductive metals. The first reaction layer 102 and the second reaction layer of present invention may further contain a moisturizer, such as cellulose, hydroxyethyl cellulose, polyethanol, polyvinyl alcohol, ethylene polymer, pyrrolidone, or gelatin and the like. The nitrite ion in the fluid sample undergoes an electrochemical reaction with p-aminobenzenesulfonamide, N-1-naphthylethylenediamine and a conductive metal to produce an electrical change.

Wherein in one of the preferred examples, the materials of the hydrophilic filter membrane can be glass fiber or cotton fiber, such as polyether (PES), polyvinylidene fluoride (PTFE), polyacrylonitrile (PAN), mixed fiber filter (MCE), glass fiber filter and the average thickness is between 0.2 to 0.4 mm, and the pore size is between 0.5 and 3 μm.

In practical operation, first, taking an adequate amount of a fluid sample, such as urine, blood, drinking water, milk, vegetable or other fluids, adding the fluid onto the hydrophilic filter membrane to filter out the impurities in the fluid sample to minimize the interference from the impurities, the filtrate will then react with aminobenzenesulfonamide of the second reaction layer 102 and undergo a chemical reaction before further reacting with the conductive metal and N-1-naphthylethylenediamine of the second reaction layer 103, wherein aminobenzenesulfonamide can react with the nitrite ions in the fluid sample and form a diazonium salt, electron ionization will occur after dissolving the diazonium salt and generate strong conductivity, and will then undergo the coupling reaction with N-1-naphthylethylenediamine. The chemical reaction is as follows:

C₆H₈N₂O₂S+NO₂⁻ [symbol] C₆H₆N₃O₂S⁺+2H₂O

C₆H₆N₃O₂S⁺+C₁₂H₁₄N₂→C₁₈H₁₉N₅O₂S+H⁺

When the nitrite ions in the fluid sample undergoes the electrochemical reaction with aminobenzenesulfonamide, the conductive metal and N-1-naphthylethylenediamine, an electric change will be generated in the sensing region. The signal of the electric current or electrical resistance is detected and delivered to the electrochemical detection module for calculation to give the nitrite concentration in the fluid sample.

According to the data analysis, the nitrite detection device provided in present invention, wherein the limit of detection (L.O.D.) of nitrite concentration of the electrochemical strip is approximately 1.39×10⁻⁷ M and the limit of detection (L.O.D.) of nitrite concentration of the colorimetric test paper is approximately 1.3×10⁻⁵ M.

When measuring nitrite concentration by using the nitrite detection device of present invention, a semi-quantitative result and a quantitative result can be obtained together and the detection is more accurate by comparing the quantitative result to the color corresponding to the concentration obtained from semi-quantitative analysis. In addition, the reaction time of said colorimetric test paper is shorter and a test result can be obtained fast, whereas the electrochemical test strip has better sensitivity and specificity, which minimizes the interference from irrelevant impurities by pre-filtering the fluid sample with a hydrophilic filter membrane and the result is determined based on the electrical signals such as the electric current or electrical resistance generated by the electrochemical reaction, not base on the color produced after completion of the reaction and therefore can avoid miss interpretation caused by the color of the fluid sample. The electrochemical test strip is easy to easy when compared to optical quantification and chromatographic quantification and no expensive equipment is required and no tedious operating procedures. Moreover, said electrochemical test strip is a direct measurement method, said detection device can be placed in the lining of sanitary pads, panties or diaper for young children or elderly people for real-time detection of nitrite ions in the urine of young children or elderly people for early detection of urinary infection.

The foregoing detailed description of the invention and the specific examples are provided herein for the purpose of illustration only, and the invention is not limited to the preferred embodiments shown. It should be understood that any changes or modifications within the spirit of the invention shall be included in the scope of present invention.

In conclusion, the nitrite detection device disclosed in present invention has been confirmed to be useful and the use of its technical means is also novel, in addition, the efficacy is apparently consistent with the design purposes. Therefore, the application is herein submitted for your examination. Please kindly review and approve this patent application.

What is claimed is:

1. A nitrite detection device comprising of:
an electrochemical test strip and a colorimetric test paper, said electrochemical test strip is installed in the electrochemical measurement region of the nitrite detection device, said colorimetric test paper is installed in the visual colorimetric region of the nitrite detection device, wherein
said electrochemical test strip is consisting of:
an insulating substrate which has a first surface and a second surface said second surface is on the back of the first surface;
an electrochemical detection structure which is installed on the first surface of said insulating substrate and is consisting of a detecting region, a sensing region and a conductive region located between the detecting region and the sensing region;
a first reaction layer which covers the sensing region and is coated with a first reactive material, said first reactive material is consisting of a buffer solution and a nitrite reactive substance, and
a second reaction layer which covers the sensing region and is coated with a second reactive material, said second reactive material is consisting of a surfactant and an electron transport material,
A fluid enters the sensing region and reacts with the first reactive material of the first reaction layer and undergoes an electrochemical reaction and then enters the sensing region via the conductive region to react with the second reactive material of the second reaction layer and undergoes an electrochemical reaction, detecting the change of the electric signals caused by the product generated from the electrochemical reaction and calculate the nitrite concentration in the fluid sample;
said colorimetric test paper is consisting of:
a sample test paper, a negative control region and a positive control region, wherein
said sample test paper is used for detection of the same fluid sample used for the electrochemical test strip, said negative control region represents colorization of a substance that does not contain nitrites, said positive control region represents colorization of a substance containing nitrites.

2. The device as recited in claim 1, wherein the insulating substrate is selected from the group consisting of polycarbonate, polyester, polyether, polyamide, polyurethane, polyimide, polypropylene, polyethylene, polyvinyl chloride, glass, glass fiber, cotton fiber, Silicon and aluminum dioxide.

3. The device as recited in claim 1, wherein the buffer solution is selected from the group consisting of phosphate, citrate and acetate buffer solution.

4. The device as recited in claim 1, wherein the surfactant is selected from the group consisting of Tween-20, Tween-80, Triton X-100, Mega8 and Polyvinylpyrrolidone (PVP).

5. The device as recited in claim 1, wherein the nitrite-reactive substance is p-aminobenzenesulfonamide.

6. The device as recited in claim 1, wherein the electron transport materials are conductive metal and N-1-naphthylethylenediamine.

7. The device as recited in claim 6, wherein the conductive metal is selected from the group consisting of gold, silver and copper.

8. The device as recited in claim 1, wherein the electrochemical test strip may further comprise an electrode structure, the electrode structure comprise a working electrode and a reference electrode covering the first surface of the insulating substrate, the working electrode and the reference electrode are conductive colloids and said conductive colloid are selected from the group consisting of copper colloid, nickel colloid, silver colloid, gold colloid, platinum colloid, silver-carbon colloid and carbon colloid.

9. The device as recited in claim 8, wherein the working electrode is gold, silver, copper, platinum or graphite and said reference electrode is a calomel electrode or a silver/silver chloride electrode.

10. The device as recited in claim 1, wherein the electrochemical test strip further comprises a hydrophilic filter membrane covering the detecting region and is used for filtration of the fluid sample.
